(19) United States
(12) Patent Application Publication
(10) Pub. No.: US 2025/0228448 A1
(43) Pub. Date: Jul. 17, 2025

(54) CAPSULE ENDOSCOPE AND ADJUSTING METHOD

(71) Applicants: Ankon Medical Technologies(Shanghai) Co., LTD, Shanghai (CN); ANX IP HOLDING PTE. LTD., SG (SG)

(72) Inventors: Xiaodong DUAN, Plano, TX (US); Shaobang ZHANG, Shanghai (CN)

(73) Assignees: Ankon Medical Technologies(Shanghai) Co., LTD, Shanghai, CN (CN); ANX IP HOLDING PTE. LTD., SG, SG (SG)

(21) Appl. No.: 18/725,815

(22) PCT Filed: Dec. 29, 2022
(86) PCT No.: PCT/CN2022/143486

§ 371 Date: Jul. 1, 2024

(30) Foreign Application Priority Data
Dec. 31, 2021 (CN) 202111673329.0

(51) Int. Cl. A61B 1/12; A61B 1/00; A61B 1/04

(52) U.S. Cl. CPC A61B 1/128; A61B 1/00034; A61B 1/041

(57) ABSTRACT

The present invention provides a capsule endoscope and an adjusting method. The capsule endoscope includes: an enclosure, the enclosure including an enclosure main body portion and enclosure end portions; an image acquisition component and an information transmission component, disposed in the enclosure end portions; a circuit processing component, disposed in the enclosure main body portion; accompanying spaces formed in the enclosure and a heat storage material is filled in the accompanying spaces; a battery and a heating component are connected to the circuit processing component, the heating component heats the battery. The adjusting method for a capsule endoscope comprises: checking the output power of the battery of the capsule endoscope; when the output power is less than a threshold, heating the battery by the heating component; and when the output power is greater than the threshold, stopping heating by the heating component.

### FIELD OF INVENTION

The present invention relates to a capsule endoscope and an adjusting method.

### BACKGROUND

Capsule endoscope, depending on its high reliability and safety, has become an effective device for the diagnosis of gastrointestinal diseases and has obtained high recognition in international medical device field. A capsule endoscope includes a complementary metal oxide semiconductor (abbreviated as CMOS) image sensor, an optical system, a battery, a transmission circuit and an antenna, etc. Images of the gastrointestinal tract of a human subject are formed on the surface of the CMOS image sensor through the optical system. The CMOS image sensor converts the optical signals into electrical signals, which are then modulated and amplified by the transmission circuit and transmitted through the antenna. These signals are received by an external receiving device and subsequently displayed on a display device. Based on the displayed images, a physician can make a diagnosis of gastrointestinal diseases for the subject in a state of painless and non-invasive gastrointestinal peristalsis.

The batteries used in capsule endoscopes are primarily silver oxide button batteries, also known as silver-zinc batteries or zinc-silver oxide batteries, which utilize silver oxide as the positive electrode, zinc as the negative electrode, and an alkaline solution as the electrolyte. These batteries are characterized by stable discharge and a high energy-to-weight ratio. Similarly, to reduce costs, some capsule endoscopes also use other alkaline button batteries. However, the examination time of a capsule endoscopy procedure is relatively long. After ingestion, the capsule is excreted through gastrointestinal peristalsis, and the entire gastrointestinal examination typically takes more than 8 hours to complete. This necessitates that the capsule endoscope's battery can provide long-lasting power. Nonetheless, silver oxide batteries and other alkaline button batteries exhibit a decrease in discharge power after a period of discharge. Therefore, improving the battery's endurance has become an urgent technical issue that needs to be addressed. Moreover, capturing images within the gastrointestinal tract requires high power and large peak currents to meet the imaging demands. Hence, enhancing the battery's ability to supply peak current is also a technical problem that needs to be resolved.

### SUMMARY OF THE INVENTION

In order to solve the problems in the prior art, the present invention provides a capsule endoscope and an adjusting method, wherein:

A capsule endoscope, comprising:
an enclosure, the enclosure comprising an enclosure main body portion and enclosure end portions;
an image acquisition component, a circuit processing component, an information transmission component, and a battery arranged within a space defined by the enclosure, where the image acquisition component, the circuit processing component and the information transmission component are electrically connected to the battery, the image acquisition component and the information transmission component are disposed in the spaces corresponding to the enclosure end portions, and the circuit processing component and the battery are disposed in the space corresponding to the enclosure main body portion; and
a heating component, the heating component is wound around the outer peripheral surface of the battery.

Optionally, the heating component is powered by the battery.

Optionally, the circuit processing component can short-circuit the battery for a certain period.

A capsule endoscope, comprising:
an enclosure, the enclosure comprising an enclosure main body portion and enclosure end portions;
an image acquisition component, a circuit processing component, an information transmission component, and a battery arranged within a space defined by the enclosure, where the image acquisition component, the circuit processing component and the information transmission component are electrically connected to the battery, the image acquisition component and the information transmission component are disposed in the spaces corresponding to the enclosure end portions, and the circuit processing component and the battery are disposed in the space corresponding to the enclosure main body portion;
at least one accommodating space is formed between the enclosure main body portion and the enclosure end portions, and the accommodating space is filled with heat storage material. Optionally, the capsule endoscope further comprises a heating component, and the heating component is wound around the outer peripheral surface of the battery.

Optionally, the heat storage material has a specific heat capacity greater than 0.7 kJ/(kg·° C.) or a phase change latent heat in 90 KJ/kg-500 KJ/kg, and a phase change temperature in 15° C.-75° C.

Optionally, the heat storage material includes one or more of paraffin, fatty acid, polyethylene, fatty alcohol, fat, microcapsule composite material, or nanocapsule material.

Optionally, the heat storage material, the circuit processing component, and the battery are connected by a thermally conductive plate.

Sealing partitions are arranged between the enclosure main body portion and the enclosure end portions, and the partitions divide the internal space of the capsule endoscope into a first accommodating space, a second accommodating space, and a third accommodating space. The second accommodating space is located between the first accommodating space and the third accommodating space. The heat storage material filled in the first accommodating space and the third accommodating space has a specific heat capacity greater than that of the heat storage material filled in the second accommodating space, or has a phase change latent heat greater than that of the heat storage material filled in the second accommodating space.

A thermal insulation layer is arranged in the first accommodating space and the third accommodating space near the inner wall of the capsule.

The capsule endoscope further comprises a thermally conductive plate. The heat storage materials in the first accommodating space, the second accommodating space and the third accommodating space are connected via the thermally conductive plate.

The heat storage material has a specific heat capacity greater than 0.7 kJ/(kg·° C.) or a phase change latent heat in 90 KJ/kg-500 KJ/kg, and a phase change temperature in 15° C.-75° C.

The enclosure main body portion of the capsule endoscope is provided with a thermal insulation layer, and the thermal insulation layer is attached to the inner wall of the enclosure, protruding towards the circuit processing component, or embedded in the enclosure main body portion and flush with the inner wall surface of the enclosure main body portion.

Optionally, the capsule endoscope further comprises a wireless charging component, the wireless charging component is electrically connected to the circuit processing component.

An adjusting method for the capsule endoscope, comprising:
Step S1, monitoring the output power of the battery of the capsule endoscope;
Step S2, heating the battery by the heating component when the output power is less than a threshold;

Optionally, if the output power remains is less than the threshold after 5 seconds-30 seconds of heating, the heating component stops heating.

Optionally, after stopping heating, if the output power is less than a critical threshold, the battery is controlled to short circuit for 1 seconds-5 seconds to increase the battery temperature.

Optionally, if the temperature of the inner surface of the enclosure of the capsule endoscope exceeds 40° C., the heating component stops heating.

Step S3, stopping heating by the heating component when the output power is greater than the threshold.

Optionally, before use of the capsule endoscope, the heat storage material in the capsule endoscope absorbs and stores heat.

Optionally, an external magnetic control device cooperates with the wireless charging component for charging, and the heat storage material stores the waste heat from wireless charging.

The solution provided by the present invention can effectively improve the battery discharge efficiency without increasing the size of the capsule endoscope, solves the problem that the battery is unable to drive the capsule endoscope in the middle and later stages, but still has residual power, thereby extending the endurance time of the capsule endoscope.

### BRIEF DESCRIPTION OF THE DRAWINGS

The accompanying drawings, which are included to provide a further understanding of the present invention and are incorporated in and constitute a part of the description, in conjunction with the following embodiments of the present invention serve to explain the principle of the present invention, but do not constitute a limitation of the present invention. In the drawings:

FIG. 1 is a schematic view of the structure of a capsule endoscope according to an embodiment of the present invention.

FIG. 2 is a schematic view of the structure of the capsule endoscope with accommodating spaces according to the embodiments of the present invention.

FIG. 3 is a schematic view of the structure of the capsule endoscope with accommodating spaces after being filled with heat storage material as in FIG. 2.

FIG. 4 is a diagram illustrating the structural relationship between the heat storage material and the heating component in the capsule endoscope.

FIG. 5 is a diagram illustrating the structural relationship between the heat storage material and the thermal insulation layer in the capsule endoscope as in FIG. 4.

FIG. 6 is a schematic view of the structure of the capsule endoscope with a plurality of accommodating spaces.

FIG. 7 is a schematic view of the structure of another capsule endoscope with a plurality of accommodating spaces as in FIG. 6.

FIG. 8 is a schematic view of the structure of another capsule endoscope with a plurality of accommodating spaces as in FIG. 6.

Elements in the drawings are: 1 image acquisition component;
2 circuit processing component;
3 information transmission component;
4 enclosure;
401 Enclosure end portions;
401-1 space corresponding to one end of the enclosure end portions;
401-2 space corresponding to the other end of the enclosure end portions;
402 enclosure main body portion;
404 partition;
4051 first accommodating space;
4052 second accommodating space;
4053 third accommodating space;
406 thermal insulation layer;
5 battery;
7 heating component;
8 thermally conductive plate;
10 heat storage material.

### DETAILED DESCRIPTION

To more clearly understand the objects, features, and advantages of the present invention, a detailed description is provided below in conjunction with the accompanying drawings and specific embodiments. It should be noted that, where there is no conflict, the embodiments and features described in the present invention can be combined with each other.

Many specific details are set forth in the following description to provide a thorough understanding of the present invention. However, the present invention can be practiced in other ways than those specifically described herein. Therefore, the scope of the present invention is not limited by the specific embodiments disclosed below.

Below, the embodiments of the present invention are described in detail with reference to the accompanying drawings.

### Embodiment 1

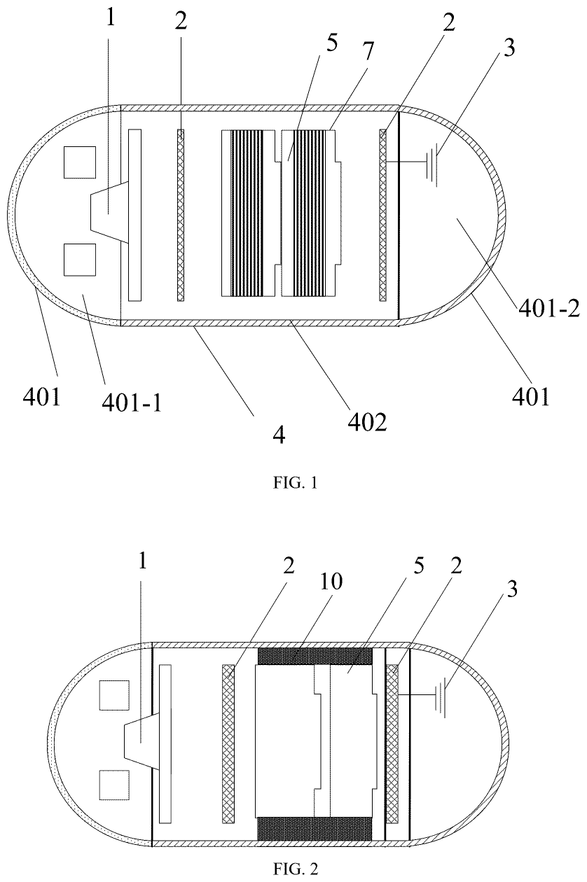

As shown in FIG. 1, a capsule endoscope, comprising: an enclosure (4), the enclosure comprising an enclosure main body portion (402) and enclosure end portions (401); an image acquisition component (1), a circuit processing component (2), an information transmission component (3), and a battery (5) arranged within a space defined by the enclosure, where the image acquisition component (1), the circuit processing component (2) and the information transmission component (3) are electrically connected to the battery (5), the image acquisition component (1) and the information transmission component (3) are disposed in the spaces (401-1, 401-2) corresponding to the enclosure end portions (401), and the circuit processing component (2) and the battery (5) are disposed in the space corresponding to the enclosure main body portion (402); and a heating component (7), the heating component (7) is wound around the battery (5). In the embodiment, the heating component is wound around the outer peripheral surface of the battery.

The image acquisition component (1) is disposed in the space (401-1) corresponding to one end of the enclosure end portions (401), the information transmission component (3) is disposed in the space (401-2) corresponding to the other end of the enclosure end portions (401), and the circuit processing component (2) is disposed in the space corresponding to the enclosure main body portion (402), with electrical connections among the three components. The image acquisition component (1) comprises or consists of a camera and light emitting diodes (abbreviated as LEDs), which can capture images of the gastrointestinal tract, and then transmit the image information to the circuit processing component (2) connected electrically. The information transmission component comprises a radio frequency (abbreviated as RF) circuit and a RF antenna. The circuit processing component (2) stores, processes, and encodes the image information, and then transmits it to the information transmission component (3) connected electrically. After being processed by the RF circuit of the information transmission component, the image information is transmitted via the RF antenna to an external magnetic control device (not shown).

The circuit processing component comprises or consists of a processor and a power management integrated circuit (abbreviated as PMIC). The processor controls all the electronic components of the capsule endoscope, while the PMIC controls the power supply to the capsule endoscope, and the PMIC is controlled by the processor.

The battery (5) supplies power to various components of the capsule endoscope, and is controlled by the PMIC, which realized control of the power supply of the battery (5).

The heating component (7), controlled by the processor, is wound around the battery (5) and can heat the battery (5).

The discharge curve of the battery shows a decrease in discharge power after a period of stable discharge, and even with the relevant circuit of Battery Management System (abbreviated as BMS) integrated in the PMIC, it is still difficult to effectively increase the discharge power in the later stage.

The capsule endoscope requires a relatively high discharge power when capturing images inside the gastrointestinal tract. When the battery is not exhausted but still has some remaining power that cannot meet the discharge power required for capturing images, and the capsule endoscope cannot capture images, the endurance time of the capsule endoscope is considered to have ended. Effectively utilizing the battery power of the capsule endoscope to capture as many images as possible is key to improving the endurance time of the capsule endoscope.

In the embodiment, the heating component (7) is wound around the casing of the battery (5) (without touching the battery electrodes), which can heat the battery when the discharge power of the battery (5) decreases (i.e., the remaining power cannot drive the capsule endoscope to capture images, but there is still remaining power to heat the heating component), to enhance the discharge capability of the battery (5), to reach the threshold for driving the capsule endoscope to capture images and extending the endurance time of the capsule endoscope.

The heating component (7) is a resistance wire made of metal material, and the heating component (7) is wound around the battery casing as fully as possible to transfer the heat generated by heating to the battery (5). The heating component (7) is connected to the circuit processing component (2). When the battery (5) cannot provide sufficient discharge power, the circuit processing component (2) controls the heating component (7) to generate heat, and the heat generated by the heating component (7) accelerates the progress of the chemical reaction inside the battery (5) to increase the discharge power to the level sufficient for capturing images.

To prevent short circuits caused by the heating component (7), the heating component (7) can also be wound around the battery casing after being covered with thermally conductive and electrically insulating material.

In another embodiment, the circuit processing component (2) can further short-circuit the battery for a certain period. In the later stage of the battery's chemical reaction, the output power significantly decreases. At this time, the battery cannot drive the capsule endoscope to capture images, and the circuit processing component controls the PMIC to briefly and periodically short-circuit the battery (5), that is, generating heat through the large current generated by the short circuit to reactivate the remaining power of the battery (5), to drive the capsule endoscope for the final capture.

### Embodiment 2

To further improve the endurance of the battery of the capsule endoscope, this embodiment provides the following capsule endoscope.

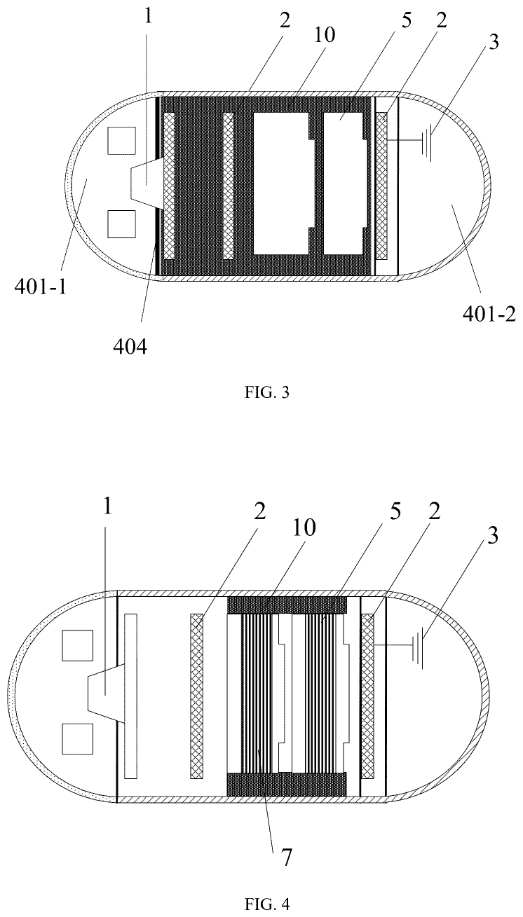

As shown in FIG. 2 and FIG. 3, a capsule endoscope, comprising: an enclosure (4), the enclosure comprising an enclosure main body portion (402) and enclosure end portions (401); an image acquisition component (1), a circuit processing component (2), an information transmission component (3), and a battery (5) arranged within a space defined by the enclosure, where the image acquisition component (1), the circuit processing component (2) and the information transmission component (3) are electrically connected to the battery (5), the image acquisition component (1) and the information transmission component (3) are disposed in the spaces (401-1, 401-2) corresponding to the enclosure end portions (401), and the circuit processing component (2) and the battery (5) are disposed in the space corresponding to the enclosure main body portion (402); and a heat storage material (10), disposed in the space corresponding to the enclosure main body portion (402).

The heat storage material can store heat in advance, and release heat to use when needed. The heat storage material provided in the capsule endoscope, which can heat the capsule endoscope before it enters the body of a subject, so that the heat storage material absorbs heat, and after the capsule endoscope enters the body of the subject, the heat storage material slowly releases the heat, which providing to the battery (5), optimizing the discharge power of the battery (5), and extending the endurance time.

The capsule endoscope operates for a long time, and the efficiency of the battery (5) mainly decreases in the middle and later stages. Selecting a material with a less thermal conductivity allows the heat storage material to release heat continuously, helping to extend the time the battery remains efficient.

The heat storage material with a high specific heat capacity, a low thermal conductivity, a large phase change latent heat, and a heat release/phase change temperature between 15° C.-75° C. is a preferred choice.

In this embodiment, the heat storage material has a specific heat capacity greater than 0.7 kJ/(kg·° C.) or a phase change latent heat in 90 KJ/kg-500 KJ/kg, and a phase change temperature in 15° C.-75° C.

Optionally, the heat storage material includes one or more of paraffin, fatty acid, polyethylene, fatty alcohol, lipid heat storage material, palmitic acid, microcapsule composite material, or nanocapsule material.

As shown in FIG. 2, in the case where the heat storage material (10) includes solid materials such as microcapsule composite materials and nanocapsule materials, etc., the heat storage material (10) including microcapsule composite materials and nanocapsule materials, etc., can be directly attached to the battery (5), and when the capsule endoscope operates in the human body, the stored heat is supplied to the battery (5) for use.

Microcapsule composite materials come in various structural forms such as single-core, multi-core, single-wall, and double-wall, etc., and are a new type of material that can be used. The structural form of nanocapsule materials is similar to that of microcapsule composite materials.

As shown in FIG. 3, in the case where the heat storage material is a liquid or gel material, such as paraffin, fatty acid, polyethylene, fatty alcohol, lipid heat storage material, palmitic acid, etc., the heat storage material can be sealed in an insulating sealed bag. The heat storage material (10) is in an amorphous structure, and the heat storage material (10) can be filled into the space corresponding to the enclosure main body portion (402), and filled into the space between the battery (5) and the enclosure, the space between the circuit processing component (2) and the enclosure, and the space between the battery (5) and the circuit processing component (2). The insulating sealed bag, which can be made of polyvinyl chloride (abbreviated as PVC) film, is not shown in FIG. 3.

The image acquisition component (1) is disposed in the space (401-1) corresponding to one end of the enclosure end portions (401), and the enclosure of the end uses a transparent material to meet the requirements of the LEDs and the camera in the image acquisition component (1) for capturing images. In the case where the heat storage material includes liquid or gel materials, a partition (404) is arranged between the space (401-1) corresponding to the enclosure end portions where the image acquisition component (1) is located and the space corresponding to the enclosure main body portion (402), to form a sealed space in the space (401-1) corresponding to the enclosure end portions where the image acquisition component (1) is located, ensuring it remains sufficiently clean to meet the requirements for capturing images.

Additionally, to enhance the shielding capability of the circuit processing component (2) and the information transmission component (3) against external electrical and magnetic interference, relevant treatments can be applied. For instance, before production and installation, the circuit processing component (2) and the information transmission component (3) can be coated with insulating glue or encapsulated with hot-pressing film.

According to this embodiment, a heat storage material is disposed in the space corresponding to the enclosure main body portion of the capsule endoscope. In the case of using an ordinary silver oxide button battery, the heat storage material can absorb and store heat before the capsule endoscope enters the human body. When the capsule endoscope enters the human body and begins to operate, the heat storage material releases the stored heat for the battery to use, effectively improving the endurance of the battery.

To further enhance the endurance of the battery, in another embodiment, as shown in FIG. 4, the heating component (7) is additionally wound around the battery (5) in the capsule endoscope. The material and structure of the heating component (7) are the same as in the Embodiment 1. In FIG. 4, only a schematic view of the structure with the heating component provided is shown when the heat storage material (10) is a solid material. A heating component can also be provided when the heat storage material is a liquid material.

In one embodiment, in a wireless charging mode, when the capsule endoscope enters the stomach for examination, the wireless charging mode is activated. At this time, the battery (5) is sufficiently charged, and the heating component (7) wound around it is heated. The heat storage material absorbs and stores the heat generated by the heating component. When the capsule endoscope moves to the intestinal tract for intestinal examination, where wireless charging is not possible, the heat storage material releases the stored heat for the battery, thereby effectively improving the endurance of the battery.

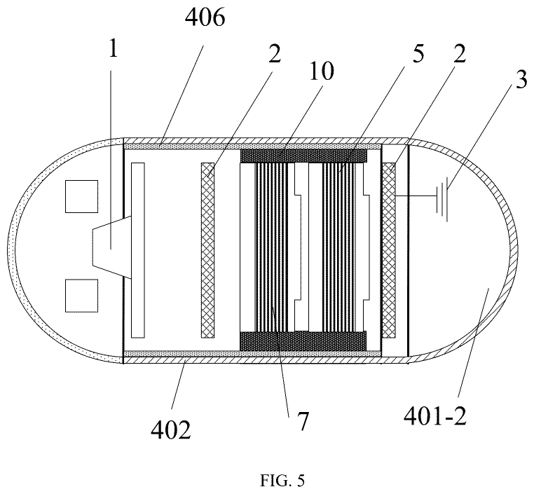

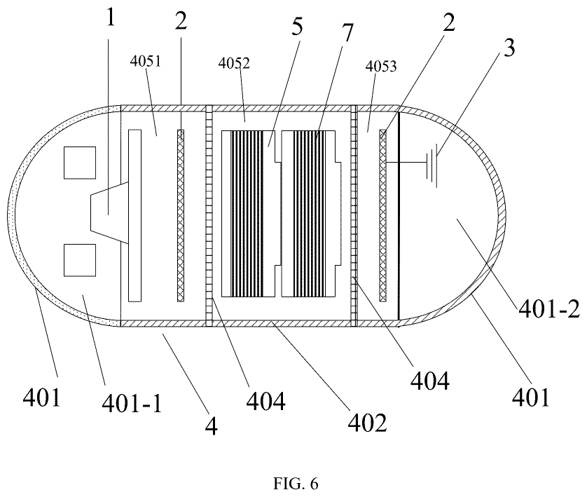

In another embodiment, as shown in FIG. 5, the enclosure main body portion (402) of the capsule endoscope is provided with a thermal insulation layer (406), and the thermal insulation layer (406) is attached to the inner wall of the enclosure, protruding towards the circuit processing component (2), or embedded in the enclosure main body portion (402) and flush with the inner wall surface of the enclosure main body portion (402). In FIG. 5, only the case where the thermal insulation layer (406) provided in the enclosure main body portion (402) is shown. The thermal insulation layer can also be provided in the inner wall of the enclosure end portion (401); the present invention is not limited to this configuration.

The thermal insulation layer (406) isolates the internal temperature from the external temperature. The heat generated by the heating component (7) disperses to the outside through the capsule endoscope enclosure. During the wireless charging phase, the heat generated by charging the capsule endoscope and by the heating component (7) can be maximally absorbed by the heat storage material. When the capsule endoscope is operating, the heat released by the heat storage material and the heat generated by the circuit processing component (2) and the battery (5) and the like during operation can be concentrated within the capsule endoscope, extending the endurance of the battery (5).

According to this embodiment of the capsule endoscope, the heat storage material is provided in the capsule endoscope, which can significantly improve the endurance of the battery. Additionally, further combine with the heating component, which can increase the heat stored in the heat storage material under the condition of wireless charging, and the heat can be used by the capsule endoscope when wireless charging is not possible, thereby heating the battery. Combined with the thermal insulation layer, heat can be concentrated within the capsule endoscope, ensuring adequate power supply to the battery during extended operation.

### Embodiment 3

To further improve the battery endurance of the capsule endoscope, this embodiment provides the following capsule endoscope based on the Embodiment 2.

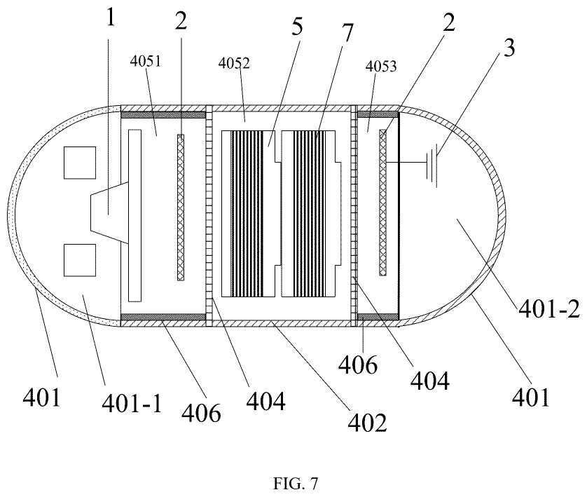

FIG. 6 is a schematic view of the plurality of accommodating spaces of the capsule endoscope. As shown in FIG. 6, the partitions (404) divide the capsule endoscope into a plurality of accommodating spaces, i.e., the first accommodating space (4051), the second accommodating space (4052) and the third accommodating space (4053). The accommodating spaces are enclosed spaces formed by the partitions (404) and the enclosure of the capsule endoscope. The second accommodating space is located between the first accommodating space and the third accommodating space. The heat storage material filled in the first accommodating space and the third accommodating space has a specific heat capacity greater than that of the heat storage material filled in the second accommodating space.

The first accommodating space (4051) is an enclosed space formed by the partition (404) and the enclosure of the capsule endoscope, excluding the remaining space occupied by the image acquisition component (1), the circuit processing component (2), and the permanent magnet (not shown).

The second accommodating space is an enclosed space formed by the partitions (404) and the enclosure of the capsule endoscope, excluding the remaining space occupied by the battery (5).

The third accommodating space is an enclosed space formed by the partition (404) and the enclosure of the capsule endoscope, or by the partition (404), the enclosure of the capsule endoscope and the enclosure end portion (401), excluding the remaining space occupied by the information transmission component (3).

The first accommodating space (4051), the second accommodating space (4052), and the third accommodating space (4053) can each be filled with the heat storage material, or partially filled with the heat storage material, or filled with the heat storage material in spaced ones, or filled with the heat storage material in a single space, or filled with different types of heat storage materials, without listing all variations herein.

Optionally, as shown in FIG. 6, only the second accommodating space (4052) is filled with an insulating heat storage material with high specific heat capacity and low thermal conductivity, or the insulating heat storage material with high phase change latent heat, while the first accommodating space (4051) and the third accommodating space (4053) are not filled with the heat storage material. In this way, the heat storage material can be preheated before examination and slowly release heat to maintain the battery (5) at an optimal operating temperature for a long time without affecting the stable operation of electronic components in the first accommodating space (4051) and the third accommodating space (4053).

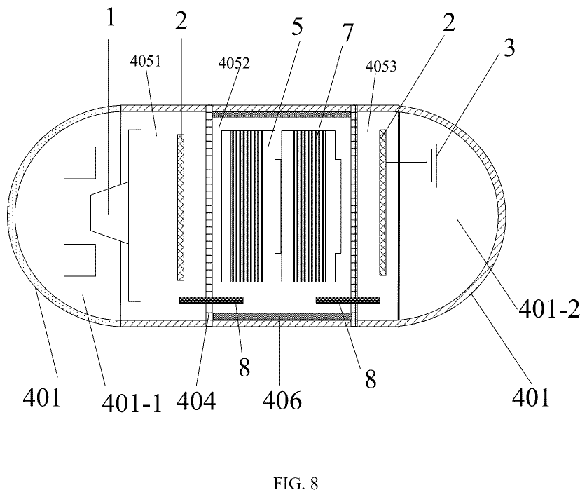

Optionally, as shown in FIG. 7, the first accommodating space (4051) and the third accommodating space (4053) are filled with the insulating heat storage materials with a high specific heat capacity and a low thermal conductivity, or the insulating heat storage materials with a high phase change latent heat, and the thermal insulation layers (406) are provided on the inner wall of the enclosure main body portion corresponding to the first accommodating space (4051) and the third accommodating space (4053). The second accommodating space (4052) can be filled with an insulating heat storage material with a high thermal conductivity. Before an examination, the capsule endoscope is preheated first, i.e., the heat storage material is preheated. When the capsule endoscope operates, the heat storage materials in the first accommodating space (4051) and the third accommodating space (4053) slowly release heat to the second accommodating space (4052). The heat storage material in the second compartment (4052), having a high thermal conductivity, can quickly heat the area around the battery, improving battery discharge efficiency. The thermal insulation layer, located at a side close to the inner wall of the capsule, can retain heat for a long time after heating, preventing excessive heat loss and extending the heating time for the capsule battery.

Optionally, as shown in FIG. 8, the first accommodating space (4051) and the third accommodating space (4053) are filled with the heat storage materials with a great specific heat capacity or a phase change temperature of 35° C.-60° C., and the second accommodating space (4052) is filled with the heat storage material with a less specific heat capacity. The first accommodating space (4051), the second accommodating space (4052), and the third accommodating space (4053) are interconnected by a thermally conductive plate (8). During operation, the heat storage materials in the first accommodating space (4051) and the third accommodating space (4053) transfer the reduced temperature or the heat released by the phase change to the second accommodating space (4052) through the thermally conductive plate (8). The heat storage material in the second accommodating space (4052), with a low thermal conductivity, slowly releases heat, persistently enhancing the efficiency of the battery (5).

In the embodiment, the heat storage materials filled, can be partially filled, or fully filled, preferably fully filled.

The embodiment uses three accommodating spaces as an example; other increases or decreases in accommodating spaces are similar and should be understood as within the scope of this solution. Additionally, filling with different types of heat storage materials and their derived variations should also be considered to be derived based on the embodiments of the present invention, and fall within the protection scope of this present invention.

### Embodiment 4

The overall working time of the capsule endoscope is relatively long, and during the middle to later stages of the examination, it is very likely that the battery (5) is insufficient to drive the capsule endoscope. To solve this problem, this embodiment discloses an adjusting method for the capsule endoscope, which comprises:
Step S1, monitoring the output power of the battery (5) of the capsule endoscope;
Step S2, heating the battery by the heating component when the output power is less than a threshold;
Step S3, stopping heating by the heating component when the output power is greater than the threshold.

In the embodiment, in the step of monitoring the output power of the battery (5) of the capsule endoscope in the adjusting method, if the output power is less than the threshold, where the threshold is set at 50% of the rated power, the battery (5) is heated. Once the output power of the battery (5) is greater than the threshold, the heating is stopped.

This method can maximize the use of the remaining power of the battery (5) for capturing images, thereby extending the endurance of the capsule endoscope.

### Embodiment 5

To further extend the endurance of the capsule endoscope, the embodiment provides another adjusting method for the capsule endoscope, which comprises:
Step S1, monitoring the output power of the battery of the capsule endoscope;
Step 2, heating the battery by the heating component when the output power is less than a threshold, where the threshold is set at 50% of the rated power; where
if, after 30 seconds of heating by the heating component, the output power is still less than the threshold, the heating component stops heating; after the heating is stopped, if the output power is less than the critical threshold, where the critical threshold is set at 10% of the rated power, the battery is controlled to short circuit for 1 second to increase its temperature; and
if the temperature of the inner surface of the enclosure of the capsule endoscope exceeds 40° C., the heating component stops heating;
Step 3, stopping heating by the heating component when the output power is greater than the threshold.

Before use of the capsule endoscope, the heat storage material in the capsule endoscope absorbs and stores heat.

In the embodiment, before performing a gastrointestinal examination, the capsule endoscope can be preheated, for example, by placing it in water at a temperature of 50° C.-95° C. After the heat storage material inside the capsule endoscope absorbs a certain amount of heat, the capsule can be swallowed along with the warm water, while completing the preparation for the examination.

During the examination in the gastrointestinal tract, the heat storage material slowly releases heat, keeping the battery (5) in a higher discharge range.

As the battery (5) continues to discharge, it may no longer be possible to use the heating component (7) to extract enough power to drive the capsule endoscope. That is, the output power of the battery (5) is less than the critical threshold. At this point, the circuit processing component (2) briefly short-circuits the battery (5). The significant heat released by the short-circuit can extract the last bit of potential from the battery (5), extending the endurance for image capturing.

During the entire examination process, the physician can use an external magnetic control device to cooperate with the wireless charging component (not shown) of the capsule endoscope for charging. In one embodiment, the wireless charging component comprises a receiving coil and a rectifier circuit. The heat storage material can store the waste heat from wireless charging through the thermally conductive plate (8).

The capsule endoscope provided by the present invention, which can improve the discharge power of the battery (5) in the middle to later stages of the examination through the provided heating component (7). Additionally, the present invention provides another capsule endoscope, which maintains the battery (5) at a higher discharge power for a long time through filling the capsule endoscope with a heat storage material, with the release and phase change heat release of the heat storage material, and combined with the heating component and thermal insulation layer.

The present invention further provides an adjusting method for the capsule endoscope, which improves the discharge power of the battery (5) in the middle to later stages of the examination through steps such as detecting the discharge power, heating by the heating component (7), and short-circuiting the battery (5).

The capsule endoscope provided by the present invention can improve battery endurance without increasing the size of the capsule endoscope, effectively enhancing battery discharge efficiency. Moreover, the capsule endoscope provided by the present invention can be used in a wireless charging mode, to solve the problem of insufficient power during wireless charging.

The above descriptions are only preferred embodiments of the present invention, and are not intended to limit the present invention. For those skilled in the art, the present invention may have various modifications and changes. Any modifications, equivalent replacements, improvements, etc. made within the spirit and principles of the present invention shall be included within the protection scope of the present invention.

1. A capsule endoscope, comprising:
an enclosure, the enclosure comprising an enclosure main body portion and enclosure end portions;
an image acquisition component, a circuit processing component, an information transmission component, and a battery arranged within a space defined by the enclosure, wherein the image acquisition component, the circuit processing component and the information transmission component are electrically connected to the battery, the image acquisition component and the information transmission component are disposed in the spaces corresponding to the enclosure end portions, and the circuit processing component and the battery are disposed in the space corresponding to the enclosure main body portion; and
a heating component, the heating component is wound around the outer peripheral surface of the battery.

2. A capsule endoscope, comprising:
an enclosure, the enclosure comprising an enclosure main body portion and enclosure end portions;
an image acquisition component, a circuit processing component, an information transmission component, and a battery arranged within a space defined by the enclosure, wherein the image acquisition component, the circuit processing component and the information transmission component are electrically connected to the battery, the image acquisition component and the information transmission component are disposed in the spaces corresponding to the enclosure end portions, and the circuit processing component and the battery are disposed in the space corresponding to the enclosure main body portion;
at least one accommodating space is formed between the enclosure main body portion and the enclosure end portions, and the accommodating space is filled with a heat storage material.

3. The capsule endoscope of claim 2, wherein the capsule endoscope comprises sealing partitions, the sealing partitions are arranged between the enclosure main body portion and the enclosure end portions, the partitions divide the internal space of the capsule endoscope into a first accommodating space, a second accommodating space, and a third accommodating space, wherein the second accommodating space is located between the first accommodating space and the third accommodating space, and the heat storage material filled in the first accommodating space and the third accommodating space has a specific heat capacity greater than that of the heat storage material filled in the second accommodating space, or has a phase change latent heat greater than that of the heat storage material filled in the second accommodating space.

4. The capsule endoscope of claim 3, wherein the capsule endoscope comprises a thermal insulation layer, the thermal insulation layer is arranged in the first accommodating space and the third accommodating space near the inner wall of the capsule endoscope.

5. The capsule endoscope of claim 4, wherein the capsule endoscope further comprises a thermally conductive plate, the heat storage materials in the first accommodating space, the second accommodating space and the third accommodating space are connected via the thermally conductive plate.

6. The capsule endoscope of claim 2, wherein the heat storage material has a specific heat capacity greater than 0.7 kJ/(kg·° C.) or a phase change latent heat in 90 KJ/kg-500 KJ/kg, and a phase change temperature in 15° C.-75° C.

7. The capsule endoscope of claim 1, wherein the enclosure main body portion of the capsule endoscope is provided with a thermal insulation layer, the thermal insulation layer is attached to the inner wall of the enclosure, protruding towards the circuit processing component, or embedded in the enclosure main body portion and flush with the inner wall surface of the enclosure main body portion.

8. An adjusting method for the capsule endoscope of any of claim 1, comprising:
Step S1, monitoring output power of the battery of the capsule endoscope;
Step S2, heating the battery by the heating component when the output power is less than a threshold;
Step S3, stopping heating by the heating component when the output power is greater than the threshold.

9. The adjusting method of claim 8, wherein before use of the capsule endoscope, the heat storage material in the capsule endoscope absorbs and stores heat.

10. An adjusting method for the capsule endoscope of claim 1, comprising:
Step S1, monitoring output power of the battery of the capsule endoscope;
Step S2, heating the battery by the heating component when the output power is less than a threshold; wherein, if the output power remains less than the threshold after 5 s-30 s of heating, the heating component stops heating;
Step S3, stopping heating by the heating component when the output power is greater than the threshold.

11. The capsule endoscope of claim 2, wherein the enclosure main body portion of the capsule endoscope is provided with a thermal insulation layer, the thermal insulation layer is attached to the inner wall of the enclosure, protruding towards the circuit processing component, or embedded in the enclosure main body portion and flush with the inner wall surface of the enclosure main body portion.

12. An adjusting method for the capsule endoscope of claim 2, comprising:
Step S1, monitoring output power of the battery of the capsule endoscope;
Step S2, heating the battery by the heating component when the output power is less than a threshold;
Step S3, stopping heating by the heating component when the output power is greater than the threshold.

13. An adjusting method for the capsule endoscope of claim 2, comprising:
Step S1, monitoring output power of the battery of the capsule endoscope;
Step S2, heating the battery by the heating component when the output power is less than a threshold; wherein, if the output power remains less than the threshold after 5 s-30 s of heating, the heating component stops heating;
Step S3, stopping heating by the heating component when the output power is greater than the threshold.
